(19) United States
(12) Patent Application Publication
(10) Pub. No.: US 2020/0113847 A1
(43) Pub. Date: Apr. 16, 2020

(54) METHODS AND FORMULATIONS FOR TREATING CHEMOTHERAPY-INDUCED NAUSEA AND VOMITING

(71) Applicant: Tilray, Inc., Seattle, WA (US)

(72) Inventor: Catherine Jacobson, Seattle, WA (US)

(21) Appl. No.: 16/598,696

(22) Filed: Oct. 10, 2019

Related U.S. Application Data
(60) Provisional application No. 62/743,839, filed on Oct. 10, 2018.

(51) Int. Cl. A61K 31/05; A61K 31/352; A61K 9/127; A61K 47/10; A61K 47/20; A61K 47/44; A61P 1/08

(52) U.S. Cl. CPC A61K 31/05; A61K 31/352; A61K 9/127; A61P 1/08; A61K 47/20; A61K 47/44; A61K 47/10

(57) ABSTRACT

The invention provides a formulation of cannabidiol and delta-9-tetrahydrocannabinol. The formulation is a solid oral dosage form. In some embodiments, the formulation is an oral liposomal formulation. The present invention also provides methods of treating chemotherapy-induced nausea and vomiting by administering such formulations to a patient before and/or after the patient receives chemotherapy treatment. The chemotherapy-induced nausea and vomiting being treated can comprise acute chemotherapy-induced nausea and vomiting, delayed chemotherapy-induced nausea and vomiting, or both.

### RELATED APPLICATIONS

This application claims the benefit of U.S. Provisional Patent Application No. 62/743,839, filed Oct. 10, 2018, the entire contents of which are incorporated herein by reference.

### TECHNICAL FIELD

This disclosure relates generally to formulations comprising a combination of cannabidiol (CBD) and delta-9-tetrahydrocannabinol (THC). The present invention also provides methods of administering such formulations to treat chemotherapy-induced nausea and vomiting (CINV).

### SUMMARY

Nausea and vomiting are common side effects of chemotherapy treatment. For chemotherapy patients, such side effects negatively affect their quality of life and their ability to comply with therapy. Certain existing products have been developed to treat CINV. However, these known products are ineffective for certain patients, have unreliable gastrointestinal absorption, poor bioavailability, and/or do not allow for rapidly self-titrating dose based on a patient's tolerance. Furthermore, existing products that include THC are provided in a high dosage strength that can cause the patient to become intoxicated.

A formulation comprising a combination of CBD and THC is described herein. The CBD and THC can be present in the formulation in an about 1:1 weight ratio. Both the THC and CBD can be obtained from Cannabis Sativa or other Cannabis plant material. The formulation can comprise a solid oral dosage form, particularly a solid oral dosage form configured to be swallowed by a patient. In some embodiments, the formulation is an oral liposomal formulation. The formulation can be provided in a dosage unit having about 2.5 mg of THC and about 2.5 mg of CBD. Advantageously, the formulation of the present disclosure provides controlled dosing and high bioavailability, while minimizing the intoxicating effects often associated with THC.

This disclosure also provides a method of treating CINV by administering to a patient such a formulation before and/or after the patient receives chemotherapy treatment. The patient can be administered the formulation in an amount such that the patient is administered between about 2.5 mg/day and about 30 mg/day of CBD and between about 2.5 mg/day and about 30 mg/day of THC. The method can be used to treat acute and/or delayed CINV.

### BRIEF DESCRIPTION OF THE DRAWINGS

FIG. 1 depicts mean plasma concentration-time plots for THC.

FIG. 2 depicts mean plasma concentration-time plots for 11-hydroxy-tetrahydrocannabinol (11-OH-THC).

FIG. 3 depicts mean plasma concentration-time plots for CBD.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Skilled artisans will recognize that the examples provided herein have many useful alternatives that fall within the scope of the invention.

As used herein, the term “patient” refers to any human or non-human mammal.

As used herein, the term “prevent” refers to completely inhibiting undesirable side effects of chemotherapy treatment. Such side effects can include nausea and vomiting. For example, preventing CINV in a patient can refer to the complete absence of both nausea and vomiting in the patient after the patient receives chemotherapy treatment.

As used herein, the term “reduce” refers to minimizing a side effect of chemotherapy treatment, such as lessening the length, intensity, and/or frequency of one or more side effect. Such side effects can include nausea and vomiting.

As used herein, the term “treat” refers to preventing or reducing undesirable side effects of chemotherapy treatment.

As used herein, the term “pharmaceutically effective amount” refers to an amount that is sufficient to treat a side effect of chemotherapy treatment. Such side effects can include nausea and vomiting. It will be understood that the pharmaceutically effective amount for a particular patient is dependent on multiple factors, including the patient's age, gender, and body weight.

As used herein, the term “nausea” refers to a disturbed and unpleasant feeling in the stomach.

As used herein, the terms “vomit” and “emesis” refer to the expulsion of stomach contents through the mouth.

As used herein, the term “retching” refers to an attempt to vomit that is not productive of stomach contents. This definition of “retching” includes “dry heaves.”

As used herein, the term “rescue therapy” refers to any medication used to relieve symptoms of nausea or vomiting. This definition excludes the formulation of the present disclosure.

As used herein, the term “no significant nausea” refers to a patient's subjective measurement of nausea of less than 2 (out of 10) on an 11-point rating scale that includes zero. In more detail, a score of zero refers to no nausea, and a score of ten refers to the worst nausea imaginable.

Disclosed herein are formulations comprising a combination of THC and CBD. The THC and CBD of the present formulation can be natural or synthetic, and can refer to any analogue, derivative, precursor, or metabolite of THC or CBD. Both the THC and CBD in the formulation can be naturally-obtained. Natural THC and CBD are obtained (e.g., extracted) from a dried Cannabis flower, such as from Cannabis Sativa or any other Cannabis flower.

The purity of both the CBD and THC in the formulation can be at least 98%. In some cases, the purity of one or both of the CBD and THC is at least 98.5%, at least 99%, at least 99.7%, or at least 99.9%.

The THC can be present in the formulation in a weight ratio of between about 0.01% and about 5.0%. In certain embodiments, the THC is present in the formulation in a weight ratio of between about 0.05% and about 2.0%. In other embodiments, the THC is present in the formulation in a weight ratio of between about 0.1% and about 1.0%, such as between about 0.25% and about 0.70% (e.g., 0.30%, 0.35%, 0.40%, 0.42%, 0.45%, 0.50%, 0.55%, 0.60%, and 0.65%).

Similarly, the CBD can be present in the formulation in a weight ratio of between about 0.01% and about 5.0%. In some embodiments, the CBD is present in the formulation in a weight ratio of between about 0.05% and about 2.0%. In still other embodiments, the CBD is present in the formulation in a weight ratio of between about 0.1% and about 1.0%, such as between about 0.25% and about 0.70% (e.g., 0.30%, 0.35%, 0.40%, 0.42%, 0.45%, 0.50%, 0.55%, 0.60%, and 0.65%).

The relative weight ratio of THC to CBD in the formulation can be from 2.0:0.5 to 0.5:2.0. In certain embodiments, the weight ratio of THC to CBD is from 1.5:1.0 to 1.0:1.5. In other embodiments, the THC and CBD are present in the formulation in an about 1:1 weight ratio.

The formulation of the present invention is provided in an oral dosage form. In some embodiments, the formulation is a solid, oral dosage form. In certain cases, the solid, oral dosage form is intended to be placed into the mouth of a patient and swallowed. Non-limiting examples of suitable dosage forms include tablets (e.g., compressed tablets and coated tablets), caplets, and capsules. In some embodiments, the formulation is provided in the form of a capsule, such as a soft gelatin capsule, a hard gelatin capsule, or a hydroxypropyl methylceullose (“HPMC”) capsule.

The solid dosage form of the present formulation is provided in the form of individual dosage units. For example, where the dosage form is a capsule, one capsule is considered an individual dosage unit. Each dosage unit has a quantity of THC and CBD sufficient to achieve a desired treatment result in certain patients. For other patients, multiple dosage units (e.g., multiple capsules) may be required to achieve the same (or different) desired treatment result. Advantageously, the dosage strength of the present formulation provides patients with better control over dosing. For example, a particular patient can take more or fewer dosage units depending on how well that patient tolerated a previous dosage amount.

In some cases, the dosage unit of the formulation comprises between about 0.5 mg and about 5.0 mg of THC, and comprises between about 0.5 mg and about 5.0 mg of CBD. For example, the dosage unit can comprise between about 1.0 mg about 4.0 mg of THC (e.g., between about 1.5 mg and about 3.5 mg of THC, or between about 2.0 mg and about 3.0 mg of THC). The dosage unit can similarly comprise between about 1.0 mg about 4.0 mg of CBD (e.g., between about 1.5 mg and about 3.5 mg of CBD, or between about 2.0 mg and about 3.0 mg of CBD). In some embodiments, each dosage unit of the formulation comprises about 2.5 mg of THC and about 2.5 mg of CBD.

For many applications, the formulation of the present invention comprises one or more pharmaceutically acceptable excipients. The term “excipient” is used herein in accordance with its plain and ordinary meaning to refer to any inactive substance that serves as the vehicle or medium for an active ingredient that helps deliver the active ingredient to a patient's system. Such excipients can comprise, but are not limited to, solubilizers, surfactants, carrier oils, and stabilizers. Since THC and CBD are oily substances that are insoluble in water, the formulation can be provided with these (or other) types of excipients, at least some of which will increase the solubility of the THC and CBD in the formulation.

In some cases, the formulation is an oral liposomal formulation. Liposomal formulations comprise liposomes, which are spherical vesicles having at least one lipid bilayer. In such embodiments, the active ingredients (i.e., the THC and CBD) are encapsulated in liposomes. Advantageously, such formulation is designed to provide more consistency in absorption, onset of action, and time to peak plasma levels (Tₘₐₓ) as compared with other conventional Cannabis formulations. The liposome-encapsulated material can then be put into a capsule.

In such embodiments, the liposomes may be any pharmaceutically acceptable material derived from soybeans or eggs. For example, the liposomes of the present formulation can comprise any lipid- or phospholipid-based liposomes, including but not limited to, phosphatidylethanolamine, phosphatidylglycerol, phosphatidylserine, phosphatidylinitisol, phosphatidylcholines, and any combinations thereof. In some cases, the lipid shell of the liposome may be or comprise lecithin, such as lysophosphatidyl choline or PHOSPHOLIPON 85G (manufactured by Phospholipid GmbH, Cologne, Germany). In addition or alternatively, the lipid component can comprise cholesterol. Suitable liposomes are well-known in the art and any such liposomes can be selected for use in the present formulation.

In various embodiments, the formulation of the present invention comprises a solubilizer. The solubilizer increases the solubility of other components in the formulation, including, e.g., THC and CBD. The solubilizer can comprise any pharmaceutically acceptable solubilizer, including, but not limited to, alkane diols and triols (e.g., glycerol or propylene glycol), polyethylene glycols (e.g., PEG 300 or PEG 400), glycol ethers (e.g., diethylene glycol monoethyl ether), or short chain-alcohols (e.g., benzyl alcohol or ethanol). Other suitable solubilizers are well-recognized in the art and can be used instead of, or in combination with, any of the above-listed solubilizers. In embodiments where the solubilizer is ethanol, this has the added benefit of enabling smaller micelle sizes. The solubilizer can be present in the formulation in a weight ratio of from about 0.05% to about 10%, such as from about 0.50% to about 5.0%; from about 0.75% to about 3.0%; or from about 1.0% to about 2.0%.

In some embodiments, the formulation comprises one or more surfactants. When provided, surfactants increase the solubility of THC and CBD in the formulation. The one or more surfactants also help provide stability and adequate dispersion for the formulation. An example of a surfactant for use in the present formulation is tocopheryl polyethylene glycol succinate 1000 (“TPGS”). TPGS is a Vitamin-E derivative of polyethylene glycol that appears to have a better safety profile than polysorbates and brings additional antioxidant activity to the formulation. Another example of a surfactant that can be used in the formulation is a lecithin, such as PHOSPHOLIPON 85G, which is phosphatidylcholine fractionated from soy lecithin (e.g., lecithin fraction enriched with phosphatidylcholine). Advantageously, phosphatidylcholine serves as a beneficial source of choline and phospholipids. Other suitable surfactants are well-known in the art and can be selected including, without limitation, polysorbates (e.g., polysorbate 20, polysorbate 60, polysorbate 85 and the like), sodium lauryl sulfate, a polyoxyethyl hydroxyl stearate, lauroyl polyoxyl 32 glyceride, propylene glycol caprylate or a phosphatidic acid derivative thereof, poloxamers, and ethoxylated vegetable oils. Any of the surfactants listed in this paragraph can be used in the formulation alone or in combination with any other surfactant. Each surfactant can be present in the formulation in an individual weight ratio of from about 4.0% to about 12.0% (e.g., from about 4.5% to about 9%; from about 5.0% to about 8.5%; or from about 6.0% to about 8.0%).

In some embodiments, the formulation comprises a carrier oil. Suitable carrier oils include, but are not limited to, olive oil, canola oil, soybean oil, coconut oil, palm oil, eucalyptus oil, lavender oil, and orange oil. In certain embodiments, the carrier oil is orange oil, which is made up of approximately 90% limonene (a terpene naturally found in Cannabis). The carrier oil can be present in the formulation in a weight ratio of from about 0.05% to about 5% (e.g., from about 0.5% to about 4%; from about 1.0% to about 3.0%; or from about 1.2% to about 2.4%).

In certain embodiments, the formulation comprises a stabilizer. The stabilizer helps provide proper dispersion of the formulation. In certain embodiments, the stabilizer is glycerin. Other suitable stabilizers that can be used instead of, or in addition to, glycerin include guar gum, xyanthan gum, sodium edatate, citric acid, sodium hyaluronate, sodium alginate acid, dextran, cellulose, hyaluronic acid, polyvinyl alcohol, polyvinyl pyrrolidone (PVP), alginate, chondritin sulfate, polygamma glutamic acid, gelatin, and chitisin. Other suitable stabilizers are well-known in the art and can be selected accordingly for use in the present formulation. The stabilizer can be present in the formulation in a weight ratio of from about 60% to about 90% (e.g., from about 65% to about 85%; from about 70% to about 82%; or from about 72% to about 78%).

In some cases, the formulation can be anhydrous. This is particularly true where the formulation is provided in the form of a capsule that comprises CBD, THC, a surfactant, a co-surfactant, a carrier oil, a solubilizer, and a stabilizer. In such embodiments, the formulation defines an anhydrous, encapsulated self-emulsifying drug delivery system.

The present invention further provides a method of preventing or reducing CINV in a patient by administering the formulation to the patient before and/or after the patient receives chemotherapy treatment. CINV broadly describes various types of nausea and vomiting that can occur in cancer patients receiving chemotherapy treatment.

As discussed above, the present formulation provides a combined dosage of THC and CBD. THC is known to provide many therapeutic properties, including antiemetic effects. However, THC is also known to provide a psychoactive high, which is undesirable in the context of treatment. By providing the present formulation as a combined dosage form, it is believed that the THC can control nausea and vomiting, while the CBD can moderate or inhibit the psychoactive effects of THC.

As used herein, reducing CINV can refer to mitigating only one side effect of chemotherapy (e.g., either nausea or vomiting) or can refer to mitigating both nausea and vomiting. In some cases, to determine whether CINV has been reduced for a particular patient, the extent of side effects for nausea and/or vomiting following one course of chemotherapy treatment can be compared to the extent of nausea and/or vomiting that occurred for the same patient after that patient received a previous course of chemotherapy. In other cases, CINV is considered to be reduced in a patient where the patient experiences no vomiting, no nausea, or no significant nausea (as defined above).

In some embodiments, the method is used to prevent or reduce acute CINV in a patient. Acute CINV refers to the onset of nausea and/or vomiting within 24 hours after administering chemotherapy treatment to the patient. In some cases, the symptoms of acute CINV occur within minutes after administering chemotherapy treatment to the patient. In other cases, the symptoms of acute CINV do not occur until hours after administering the chemotherapy treatment. Often, the symptoms of acute CINV peak after about six hours following chemotherapy treatment and can last for approximately 24 hours.

In certain other embodiments, the method is used to prevent or reduce delayed CINV in a patient. Delayed CINV refers to the onset of nausea and/or vomiting 24 hours or later after administering chemotherapy treatment to the patient. In some cases, delayed CINV can last for several days. As used herein, delayed CINV refers to the onset of nausea and/or vomiting within 24-120 hours of receiving chemotherapy treatment.

In some cases, the method is used to treat delayed CINV and acute CINV in a patient. In such instances, the formulation is administered to the patient before and/or after the patient receives chemotherapy treatment in order to prevent or reduce the onset of nausea and vomiting at any point within 120 hours after the patient receives the chemotherapy treatment. In many cases, when treating both delayed and acute CINV, the formulation will be administered to the patient both before and after the patient receives chemotherapy treatment.

In certain embodiments, the method comprises administering the formulation to the patient before and/or after the patient receives emetogenic chemotherapy treatment. The formulation can be administered to a patient who has previously experienced CINV, or can be administered to a patient who will be receiving emetogenic chemotherapy treatment for the first time.

As used herein, the term “emetogenicity” refers to the tendency of a chemotherapy agent to cause nausea and/or vomiting. In particular, highly emetogenic agents refer to medications or doses that cause CINV in >90% of patients; moderately emetogenic agents refer to medications that induce CINV in 30% to 90% of patients; low emetogenic agents refer to medications that are associated with CINV rates of 10% to 30%; and minimally emetogenic agents refer to medications that cause CINV in <10% of patients.

Drugs associated with moderate and high emetic risk are identified in the 2016 MASCC/ESMO Antiemetic Guideline. Based on this guidance, drugs with high emetic risk include: anthracycline/cyclophosphamide combination; carmustine; cisplatin; cyclophosphamide when administered at greater than 1500 mg/m²; dacarbazine; mechlorethamine; and streptozocin. Drugs classified as moderate emetic risk include: alemtuzumab; azacitidine; bendamustine; carboplatin; clofarabine; cyclophosphamide when administered at less than 1500 mg/m², cytarabine when administered at less than 1000 mg/m²; daunorubicin; doxorubicin; epirubicin; idarubicin; ifosfamide; irinotecan; oxaliplatin; romidepsin; temozolomide IV; thiotepa; and trabectedin.

In many cases, the method of the present invention is used before and/or after the patient receives moderate-to-highly emetogenic intravenous chemotherapy. As indicated above, based solely on the type of chemotherapy agent, patients undergoing moderate-to-highly emetogenic chemotherapy treatment are in general more likely to experience CINV than are patients receiving low or minimal emetogenic chemotherapy treatment. However, it should be understood that the present method can also be used before and/or after a patient receives minimal or low emetogenic chemotherapy treatment.

In particular, the present method involves administering pharmaceutically effective amounts of THC and CBD for treating CINV. It will be understood that the pharmaceutically effective amounts for a given patient are variable. In some cases, the pharmaceutically effective amounts of each of THC and CBD can be as high as 5 mg/day, as high as 10 mg/day, as high as 15 mg/day, as high as 20 mg/day, or even as high as 30 mg/day. Accordingly, in some embodiments, the pharmaceutically effective amount of CBD comprises between about 2.5 mg/day and about 30 mg/day, and the pharmaceutically effective amount of THC comprises between about 2.5 mg/day and about 30 mg/day. For example, the pharmaceutically effective amount of CBD can be, but is not limited to, between about 2.5 mg/day and about 5 mg/day, between about 2.5 mg/day and about 7 mg/day, between about 2.5 mg/day and about 10 mg/day, between about 2.5 mg/day and about 12.5 mg/day, between about 2.5 mg/day and about 15 mg/day, between about 2.5 mg/day and about 17.5 mg/day, between about 2.5 mg/day and about 20 mg/day, between about 2.5 mg/day and about 22.5 mg/day, between about 2.5 mg/day and about 25 mg/day, between about 2.5 mg/day and about 27.5 mg/day or between about 2.5 mg/day and about 30 mg/day. Similarly, the pharmaceutically effective amount of THC can be, but is not limited to, between about 2.5 mg/day and about 5 mg/day, between about 2.5 mg/day and about 7 mg/day, between about 2.5 mg/day and about 10 mg/day, between about 2.5 mg/day and about 12.5 mg/day, between about 2.5 mg/day and about 15 mg/day, between about 2.5 mg/day and about 17.5 mg/day, between about 2.5 mg/day and about 20 mg/day, between about 2.5 mg/day and about 22.5 mg/day, between about 2.5 mg/day and about 25 mg/day, between about 2.5 mg/day and about 27.5 mg/day, or between about 2.5 mg/day and about 30 mg/day.

It will be understood that the pharmaceutically effective amounts of THC and CBD can be increased or reduced within the above-noted ranges, depending on the patient's tolerance to the treatment. For example, if a patient becomes intoxicated as a result of receiving 30 mg/day of THC, that patient can receive a lower dose of THC for the next dosage to avoid or reduce the intoxicating effects experienced at the higher dose.

The formulation can be administered in one or more doses per day on any (e.g., every) day the patient receives chemotherapy treatment. In some cases, the formulation is administered only once per day. In other cases, the formulation is administered in multiple doses per day, such as two times per day, three times per day, four times per day, or five times per day. In some embodiments, the formulation is administered to the patient three times per day every day that the formulation is administered.

In certain embodiments, a patient is administered the formulation one or more days prior to receiving chemotherapy treatment. In some cases, the formulation is administered to the patient one day before the patient receives the chemotherapy treatment. In treatments of this nature, the formulation can be administered to the patient within 24 hours before the patient receives the chemotherapy treatment, such as within 22 hours, within 20 hours, within 16 hours, or within 12 hours before the patient receives the chemotherapy treatment. In other cases, one day before chemotherapy treatment can refer to a time period that is more than 24 hours before the patient receives the chemotherapy treatment. For instance, within this definition, a patient can receive the formulation early morning on one day, and then receive chemotherapy treatment late at night on the following day.

When the formulation is administered to the patient on the day before the patient receives the chemotherapy treatment, the patient can be administered between about 2.5 mg and about 12.5 mg of CBD, and between about 2.5 mg and about 12.5 mg of THC. For example, the patient can be administered about 2.5 mg, about 5 mg, about 7.5 mg, about 10 mg, or about 12.5 mg of CBD on the day before the patient receives chemotherapy. Similarly, on the day before the patient receives chemotherapy, the patient can be administered about 2.5 mg, about 5 mg, about 7.5 mg, about 10 mg, or about 12.5 mg of THC.

In addition, the formulation can be administered to the patient in a single dose or in multiple doses on the day before the patient receives the chemotherapy treatment. In certain embodiments, there are at least four hours (e.g., at least five hours, at least six hours, at least seven hours, or at least eight hours) between administering each dose on the day before the patient receives the chemotherapy treatment.

In some cases, the formulation is administered to the patient on a same day the patient receives the chemotherapy treatment. Such administration can be instead of, or in addition to, administering the formulation on one or more days prior to the chemotherapy treatment. For instance, the patient can be administered the formulation one day before chemotherapy treatment, and can be administered another dosage of the formulation on the same day as the chemotherapy treatment.

On the day the patient receives the chemotherapy treatment, the patient can be administered between about 2.5 mg and about 20 mg of CBD, and between about 2.5 mg and about 20 mg of THC. For example, on the day of chemotherapy treatment, the patient can receive about 2.5 mg, about 5 mg, about 7.5 mg, about 10 mg, about 12.5 mg, about 15 mg, about 17.5 mg, or about 20 mg of CBD. Similarly, on the same day, the patient can receive about 2.5 mg, about 5 mg, about 7.5 mg, about 10 mg, about 12.5 mg, about 15 mg, about 17.5 mg, or about 20 mg of THC.

The formulation can be administered to the patient in a single dose or in multiple doses on the day the patient receives the chemotherapy treatment. Where multiple doses are provided, at least one dose can be administered to the patient before the chemotherapy treatment, and at least one dose can be administered to the patient after the chemotherapy treatment.

Where a dose is administered to a patient on the same day as chemotherapy treatment, the patient can be administered the formulation between about thirty minutes and about four hours before the chemotherapy treatment (e.g., about thirty minutes, about one hour, about two hours, about three hours, or about four hours before chemotherapy treatment). In certain cases, the formulation is administered to the patient about one hour before chemotherapy treatment.

In some embodiments, the formulation is administered to the patient after the chemotherapy treatment on the same day the patient receives the chemotherapy treatment. In these embodiments, the formulation can be administered immediately after the chemotherapy treatment, or hours (e.g., two hours, three hours, four hours, or five hours) after the chemotherapy treatment. In some embodiments, the formulation is administered to the patient immediately after the chemotherapy treatment as well as hours after the chemotherapy treatment, such as about four hours after the chemotherapy treatment.

In certain embodiments, the formulation is administered to the patient on one or more days after the patient receives the chemotherapy treatment. This can be instead of, or in addition to, the patient being administered the formulation on the day of, and/or one or more days before, the chemotherapy treatment. In some embodiments, the formulation is administered to the patient on one or more days before the chemotherapy treatment, on the day of chemotherapy treatment, and on one or more days after the chemotherapy treatment. In certain cases, the formulation is administered to the patient every day beginning one day before the patient receives the chemotherapy treatment and up to three days after the patient receives the chemotherapy treatment. In some cases, the formulation is administered to the patient every day beginning one day before the patient receives the chemotherapy treatment and up to four days after the patient receives the chemotherapy treatment.

In certain embodiments, the patient is administered between about 2.5 mg and about 30 mg of CBD and between about 2.5 mg and about 30 mg of THC for multiple days after the patient receives chemotherapy treatment. For example, the patient can be administered about 2.5 mg, about 5.0 mg, about 7.5 mg, about 10 mg, about 12.5 mg, about 15 mg, about 17.5 mg, about 20 mg, about 22.5 mg, about 25.0 mg, about 27.5 mg, or about 30.0 mg of CBD for multiple days after the patient receives chemotherapy treatment. Similarly, the patient can be administered about 2.5 mg, about 5.0 mg, about 7.5 mg, about 10 mg, about 12.5 mg, about 15 mg, about 17.5 mg, about 20 mg, about 22.5 mg, about 25.0 mg, about 27.5 mg, or about 30.0 mg of THC for multiple days after the patient receives the chemotherapy treatment.

The above noted dosage amounts (e.g., between about 2.5 mg and about 30 mg of both CBD and THC) can be administered on the day immediately following chemotherapy treatment, on the first and second days immediately following chemotherapy treatment, or on the first, second, and third days immediately following chemotherapy treatment. On a fourth day after the patient receives the chemotherapy treatment, the patient can be administered between about 2.5 mg and about 20 mg of CBD, and between about 2.5 mg and about 20 mg of THC, including any dosage of CBD and THC that falls within these ranges.

In certain embodiments, the treatment provides the patient with a complete response. As used herein, the term “complete response” refers to a patient having no emesis and no use of rescue medications due to the chemotherapy treatment.

### Example 1

### Production of Formulation A

The required amount of THC, CBD, Vitamin E TPGS, Phospholipon 85 G, Organic Orange Sweet Essential Oil, and Ethanol are weighed out, based on Formulation A in Table 1 below. These components are then transferred sequentially into a container, allowing for the THC and CBD to fully dissolve in the lipid mixture.

Once the THC and CBD have dissolved, some of the ethanol is removed. The container is then weighed to determine if a sufficient amount of ethanol has been removed in order to ensure that the formulation is compatible with the capsule and to also ensure micelle formulation in the next step (upon dispersion in glycerin). If it is determined that sufficient ethanol has been removed, the ethanol removal process is stopped. If insufficient ethanol has been removed, the ethanol removal process continues until the target level of ethanol is achieved.

While still hot and fluid, the material is transferred to a container and the required amount of glycerin is added, based on the requirements of Formulation A. The mixture is then stirred and heated and filled into HPMC capsules.

TABLE 1
Formulation A

| | | Quantity per | |
|---|---|---|---|
| Component | Function | unit (mg) | Percent weight |
| THC | Active | 2.5 | 0.42 |
| CBD | Active | 2.5 | 0.42 |
| Vitamin E TPGS | Co-Surfactant | 40 | 6.67 |
| Food Grade | | | |
| Phospholipon 85 G | Surfactant | 40 | 6.67 |
| Organic Orange, | Carrier Oil | 8.5 | 1.42 |
| Sweet Essential | | | |
| Oil | | | |
| Ethanol, USP | Solubilizer | 6.5 | 1.08 |
| Glycerin, USP | Stabilizer | 500 | 83.33 |
| Total | | 600 | 100.0 |
| White HPMC | Capsule | 1 | |

### Example 2

### Production of Formulation B

Example 1 is repeated, except that the formulation listed below (Formulation B) in Table 2 is used instead of Formulation A.

TABLE 2
Formulation B

| | | Quantity per unit | |
|---|---|---|---|
| Component | Function | (mg) | Percent weight |
| THC | Active | 2.5 | 0.5 |
| CBD | Active | 2.5 | 0.5 |
| Vitamin E TPGS | Co-Surfactant | 40 | 8.0 |
| Food Grade | | | |
| Phospholipon 85 G | Surfactant | 40 | 8.0 |
| Organic Orange, | Carrier Oil | 10 | 2.0 |
| Sweet Essential | | | |
| Oil | | | |
| Ethanol, USP | Solubilizer | 10 | 2.0 |
| Glycerin, USP | Stabilizer | 395 | 79.0 |
| Total | | 500 | 100.0 |
| White HMPC | Capsule | 1 | |

### Example 3

### Clinical Studies

A single-dose, randomized, open-label, four-period, four-sequence, four-treatment, single-center, four-way crossover, comparative bioavailability pilot study was conducted to compare the bioavailability of Formulation B to conventional Cannabis extracts (e.g., buccal spray Cannabis formulations).

Formulation B was specifically designed to address the highly variable Tₘₐₓ for such conventional Cannabis extracts. Tₘₐₓ is an important parameter to control, as this will often determine when patients should receive a study drug.

Twelve healthy, non-smoking, occasional Cannabis users, male and non-pregnant female volunteers, 18-53 years of age, inclusive, were administered two dose levels of Formulation B, one dose level of THC and CBD (1:1 weight ratio) in grapeseed oil, and one dose level of conventional Cannabis extracts. These treatments were provided under fasting conditions in a randomized order, seven days apart. Treatment groups included:
Treatment A: 1×Formulation B capsule (THC/CBD: 2.5 mg/2.5 mg) [total dose: 2.5 mg/2.5 mg THC/CBD]
Treatment B: 2×Formulation B capsules (THC/CBD: 2.5 mg/2.5 mg) [total dose: 5 mg/5 mg THC/CBD]
Treatment C: 1×1 mL grapeseed oil containing THC/CBD (10 mg/10 mg) [total dose: 10 mg/10 mg THC/CBD]
Reference: 4×100 μL of conventional Cannabis extracts containing THC/CBD [total dose: 10.8 mg/10 mg THC/CBD]

Subjects were confined in in-patient units from at least 10 hours prior to dosing until at least 24 hours post dose, for a total of at least 34 hours for each study period. Blood sampling occurred at the following time points: Pre-dose (0 hour), and at 0.33, 0.67, 1, 1.33, 1.67, 2, 2.33, 2.67, 3, 3.5, 4, 4.5, 6, 9, 12 and 24 hours post-dose in each study period.

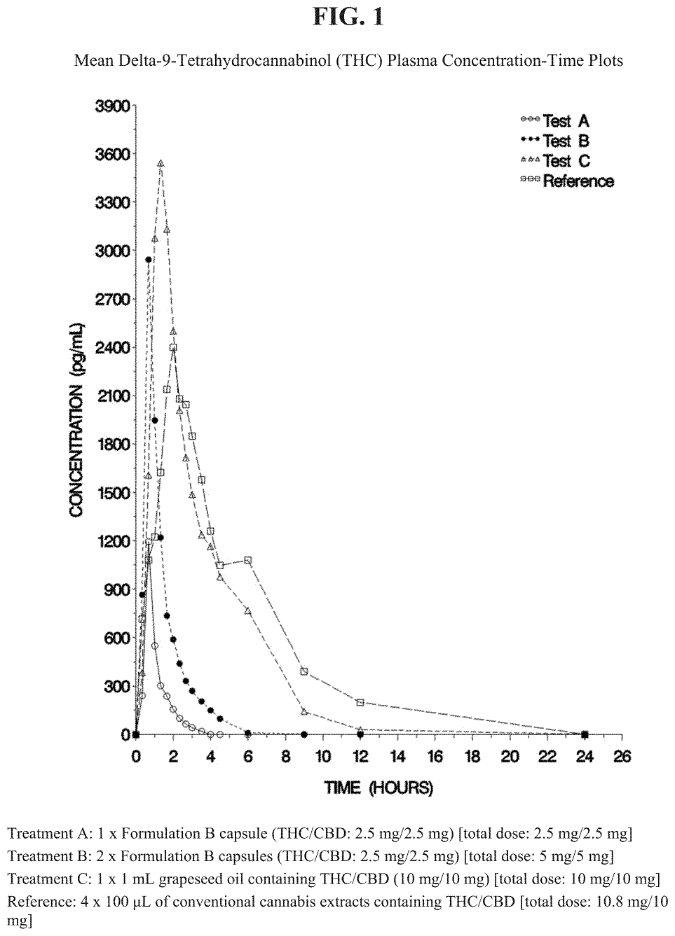

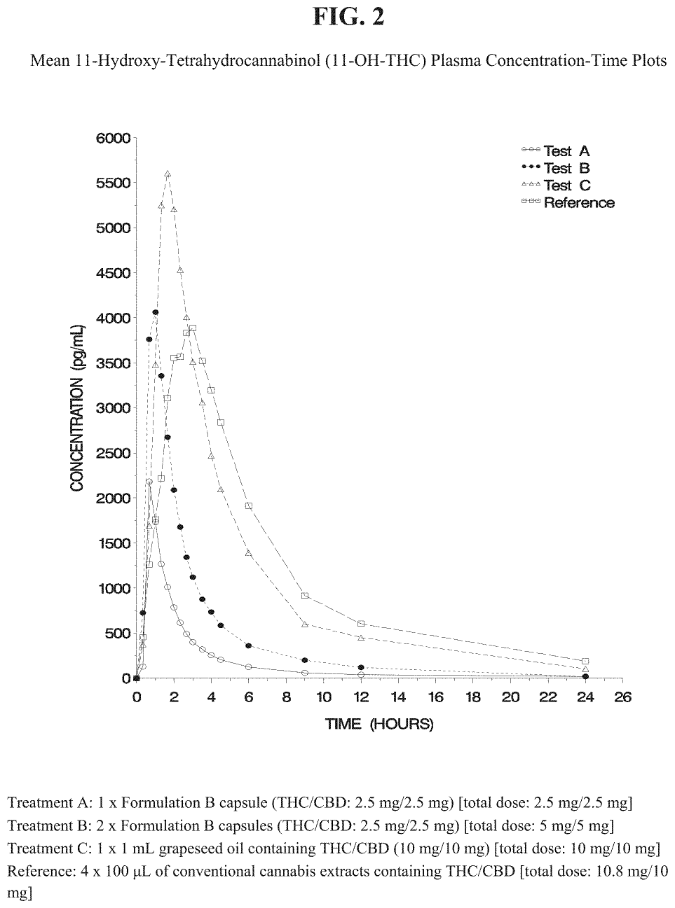

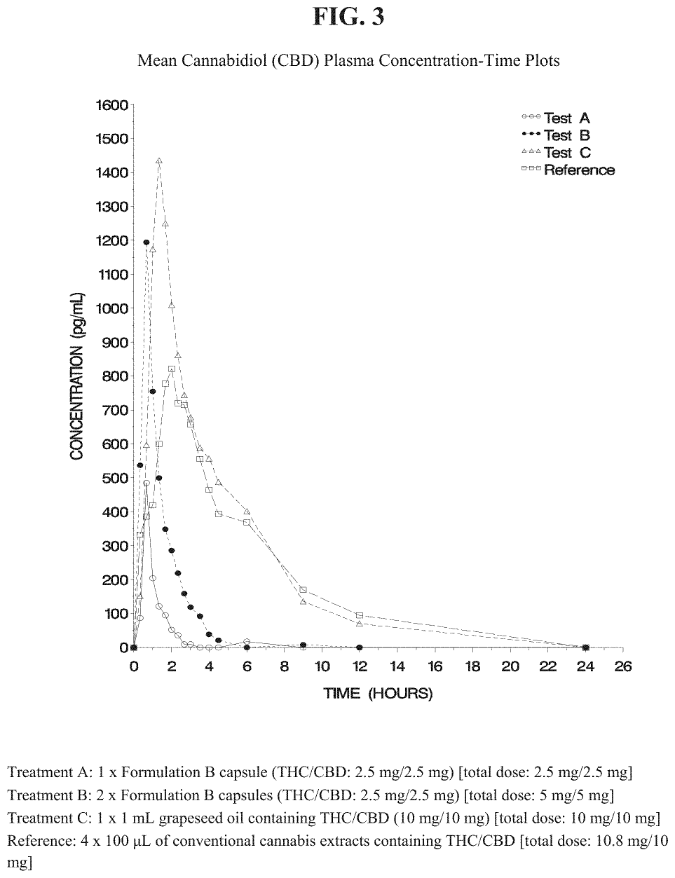

Plasma concentration-time profiles for THC, its primary metabolite 11-hydroxy-tetrahydrocannabinol (11-OH-THC), and CBD are shown in FIGS. 1, 2, and 3 respectively. In this study, as explained in greater detail below, Formulation B demonstrated a lower extent of absorption and a much higher rate of absorption of THC, 11-OH-THC and CBD, as compared to conventional Cannabis extracts.

In particular, mean maximum plasma concentrations (Cₘₐₓ) plasma levels of all analytes (THC, 11-OH-THC, and CBD) were approximately 2- to 2.5-fold higher in subjects administered two Formulation B capsules (Treatment B: total dose: 5 mg/5 mg THC/CBD), compared to conventional Cannabis extracts that were provided at approximately double the dosage (10.8 mg/10 mg THC/CBD).

In addition, the median time to maximum plasma concentration (Tₘₐₓ) of THC and CBD from Formulation B was less than half that of conventional Cannabis extracts, with peak plasma levels of THC appearing at 40 minutes post-dose for Formulation B, compared to one hour and 40 minutes for conventional Cannabis extracts.

Area under the plasma concentration-time curve from time 0 to last measurable concentration (AUC₀₋ₗₐₛₜ), a measure of overall absorption, of Formulation B (total dose: 5 mg/5 mg THC/CBD) was 60-80% that of conventional Cannabis extracts for all analytes. It should be noted that the dosage of Formulation B referred to here (5 mg/5 mg THC/CBD) is approximately half that of the dosage tested for conventional Cannabis extracts (10.8 mg/10 mg THC/CBD).

At the highest dose tested (5 mg/5 mg THC/CBD), half-lives of analytes from Formulation B were 30-60% those of analytes from conventional Cannabis extracts, indicating that active ingredients in Formulation B are cleared at about twice the rate as the active ingredients in conventional Cannabis extracts.

The mean pharmacokinetic parameters of THC for all dose groups are summarized in Table 3 below.

TABLE 3
Summary of Pharmacokinetic Parameters for Plasma THC

| | | | | Reference |
|---|---|---|---|---|
| | Treatment A | Treatment B | | (Conventional |
| | (1 × Formula B | (2 × Formula B | Treatment C | cannabis |
| | capsule) | capsules) | (THC/CBD in oil) | extracts) |
| Total Dose | 2.5 mg/2.5 mg | 5 mg/5 mg | 10 mg/10 mg | 10.8 mg/10 mg |
| (THC/CBD) | | | | |
| Cₘₐₓ (pg/mL) | 477.050 ± 214.314 | 696.180 ± 288.534 | 468.804 ± 178.527) | 291.440 ± 102.819 |
| | (200.165-831.047) | (354.460-1255.020) | (280.547-904.487) | (192.573-531.805) |
| Tₘₐₓ (hr) | 0.67 | 0.67 | 1.33 | 1.67 |
| median | (0.67-0.67) | (0.33-1.33) | (1.00-6.00) | (0.33-2.67) |
| (range) | | | | |
| AUC₀₋ₗₐₛₜ | 381.112 ± 179.177 | 669.281 ± 145.135 | 1072.255 ± 579.742 | 1068.189 ± 418.029 |
| (hr * pg/mL) | (137.290-641.787) | (347.019-863.472) | (368.788-2517.319) | (507.785-1884.047) |
| AUC_{0-∞} | 461.576 ± 182.854 | 718.259 ± 145.524 | 926.018 ± 362.205 | 1160.615 ± 428.536 |
| (hr * pg/mL) | (216.374-696.684)^{a} | (384.748-914.514) | (381.482-1507.964)^{b} | (542.071-2007.239) |
| λ (1/hr) | 0.9419 ± 0.2265 | 0.5579 ± 0.1149 | 0.6019 ± 0.1162 | 0.2550 ± 0.1014 |
| | (0.6456-1.4363)^{a} | (0.4131-0.8286) | (0.4430-0.8599)^{b} | (0.1451-0.4654) |
| t½ (hr) | 0.77 ± 0.17 | 1.29 ± 0.24 | 1.19 ± 0.21 | 3.06 ± 1.00 |
| | (0.48-1.07)^{a} | (0.84-1.68) | (0.81-1.56)^{b} | (1.49-4.78) |

^{a}N = 9
^{b}N = 10
AUC₀₋ₗₐₛₜ = area under the plasma concentration-time curve from time 0 to last measurable concentration;
CBD = cannabidiol;
Cₘₐₓ = maximum plasma concentration;
λ = terminal elimination rate constant;
THC = delta-9-tetrahydrocannabinol;
t 1/2 = terminal half-life;
Tₘₐₓ = time to Cₘₐₓ
Data presented as arithmetic mean ± SD (range) and N = 12, unless otherwise indicated.

The mean pharmacokinetic parameters for 11-OH-THC are summarized for all dose groups in Table 4 below.

TABLE 4
Summary of Pharmacokinetic Parameters for Plasma 11-OH-THC

| | | | | Reference |
|---|---|---|---|---|
| | Treatment A | Treatment B | | (Conventional |
| | (1 × Formula B | (2 × Formula B | Treatment C | cannabis |
| | capsule) | capsules) | (THC/CBD in oil) | extracts) |
| Total Dose | 2.5 mg/2.5 mg | 5 mg/5 mg | 10 mg/10 mg | 10.8 mg/10 mg |
| (THC/CBD) | | | | |
| Cₘₐₓ | 928.073 ± 625.823 | 907.279 ± 585.946 | 614.887 ± 264.408 | 413.277 ± 218.479 |
| (pg/mL) | (199.558-2192.375) | (284.731-1992.379) | (194.011-1027.958) | (143.136-886.370) |
| Tₘₐₓ (h) | 0.67 | 1.00 | 1.67 | 2.00 |
| median | (0.67-1.00) | (0.67-1.67) | (1.00-6.00) | (1.00-3.00) |
| (range) | | | | |
| AUC₀₋ₗₐₛₜ | 1615.974 ± 1016.392 | 2050.974 ± 1100.986 | 2478.045 ± 1220.232 | 2487.775 ± 1304.529 |
| (hr * pg/mL) | (482.655-3107.079) | (746.718-4182.206) | (787.651-4764.437) | (720.162-5157.252) |
| AUC_{0-∞} | 1735.859 ± 1061.532 | 2279.159 ± 1181.007 | 2635.288 ± 1269.424 | 2691.396 ± 1414.600 |
| (hr * pg/mL) | (685.508-3379.076)^{a} | (851.837-4388.820)^{a} | (836.582-5053.556) | (850.398-5631.333) |
| λ (1/hr) | 0.2935 ± 0.1143 | 0.2223 ± 0.1155 | 0.1454 ± 0.0674 | 0.1135 ± 0.0366 |
| | (0.0973-0.4674)^{a} | (0.0784-0.4299)^{a} | (0.0726-0.2539) | (0.0844-0.2139) |
| t½ (hr) | 2.86 ± 1.60 | 4.07 ± 2.27 | 5.67 ± 2.24 | 6.52 ± 1.46 |
| | (1.48-7.12)^{a} | (1.61-8.84)^{a} | (2.73-9.54) | (3.24-8.21) |

^{a}N = 11
11-OH-THC = 11-hydroxy-tetrahydrocannabinol;
AUC0-last = area under the plasma concentration-time curve from time 0 to last measurable concentration;
CBD = cannabidiol;
Cmax = maximum plasma concentration;
λ = terminal elimination rate constant;
THC = delta-9-tetrahydrocannabinol;
t½ = terminal half-life;
Tmax = time to Cmax
Data presented as arithmetic mean ± SD (range) and N = 12, unless otherwise indicated.

The mean pharmacokinetics for CBD for all dose groups are summarized in Table 5 below.

TABLE 5
Summary of Pharmacokinetic Parameters for Plasma CBD

| | | | | Reference |
|---|---|---|---|---|
| | Treatment A | Treatment B | | (Conventional |
| | (1 × Formula B | (2 × Formula B | Treatment C | cannabis |
| | capsule) | capsules) | (THC/CBD in oil) | extracts) |
| Total Dose | 2.5 mg/2.5 mg | 5 mg/5 mg | 10 mg/10 mg | 10.8 mg/10 mg |
| (THC/CBD) | | | | |
| Cₘₐₓ | 194.514 ± 117.203 | 300.731 ± 219.101 | 186.538 ± 95.223 | 117.510 ± 61.661 |
| (pg/mL) | (40.376-394.897) | (69.873-675.457) | (65.816-376.204) | (35.815-233.355) |
| Tₘₐₓ (hr) | 0.67 | 0.67 | 1.33 | 1.67 |
| median | (0.33-1.00) | (0.33-1.33) | (1.00-6.00) | (0.33-6.00) |
| (range) | | | | |
| AUC₀₋ₗₐₛₜ | 159.153 ± 149.539 | 294.183 ± 149.362 | 501.683 ± 364.808 | 419.101 ± 209.867 |
| (hr * pg/mL) | (6.864-501.700) | (73.269-497.187) | (193.369-1449.314) | (78.092-926.872) |
| AUC_{0-∞} | 280.550 ± 119.696 | 314.899 ± 114.968 | 418.610 ± 204.457 | 531.279 ± 274.959 |
| (hr * pg/mL) | (140.402-380.814)^{a} | (180.322-468.555)^{b} | (216.830-802.778)^{b} | (103.860-1096.351)^{c} |
| λ (1/hr) | 1.0299 ± 0.4872 | 0.5425 ± 0.1829 | 0.6279 ± 0.2485 | 0.2081 ± 0.1122 |
| | (0.6093-1.6396)^{a} | (0.3891-0.8801)^{b} | (0.3246-1.0126)^{b} | (0.0414-0.4367)^{c} |
| t½ (hr) | 0.79 ± 0.35 | 1.38 ± 0.38 | 1.28 ± 0.54 | 4.88 ± 4.36 |
| | (0.42-1.14)^{a} | (0.79-1.78)^{b} | (0.68-2.14)^{b} | (1.59-16.72)^{c} |

^{a}N = 4
^{b}N = 7
^{c}N = 10
AUC₀₋ₗₐₛₜ = area under the plasma concentration-time curve from time 0 to last measurable concentration;
CBD = cannabidiol;
Cₘₐₓ = maximum plasma concentration;
λ = terminal elimination rate constant;
THC = delta-9-tetrahydrocannabinol;
t½ = terminal half-life;
Tₘₐₓ = time to Cₘₐₓ
Data presented as arithmetic mean ± SD (range) and N = 12, unless otherwise indicated.

The Test/Reference ratios of geometric means, the corresponding 90% confidence intervals, and the intra-subject variability for AUC₀₋ₜ, AUC_{0-∞} and Cₘₐₓ for THC, 11-OH-THC and CBD under fasting conditions are presented in Table 6 below.

TABLE 6

| | Test/Reference Ratio of Geometric Means (90% Confidence Interval) | | | |
|---|---|---|---|---|
| | (%) | | | |
| | Treatment A | Treatment B | Treatment C | Intra- |
| Pharmacokinetic | vs | vs | vs | Subject |
| Parameter | Reference | Reference | Reference | CV (%) |

THC

| | | | | | | | |
|---|---|---|---|---|---|---|---|
| AUC₀₋ₗₐₛₜ | 33.96 | (27.32-42.20) | 65.55 | (52.74-81.47) | 94.84 | (76.30-117.88) | 30.33 |
| AUC_{0-∞} | 38.90 | (31.93-47.39) | 64.57 | (54.18-76.95) | 81.29 | (67.19-98.35) | 24.28 |
| Cₘₐₓ | 155.99 | (126.16-192.88) | 232.09 | (187.70-286.96) | 158.85 | (128.47-196.41) | 29.57 |

11-OH-THC

| | | | | | | | |
|---|---|---|---|---|---|---|---|
| AUC₀₋ₗₐₛₜ | 60.34 | (50.19-72.55) | 82.18 | (68.35-98.81) | 99.85 | (83.05-120.05) | 25.53 |
| AUC_{0-∞} | 63.82 | (53.54-76.07) | 80.97 | (67.93-96.52) | 98.24 | (82.88-116.45) | 23.51 |
| Cₘₐₓ | 204.96 | (169.64-247.65) | 202.52 | (167.62-244.69) | 152.07 | (125.86-183.74) | 26.24 |

CBD

| | | | | | | | |
|---|---|---|---|---|---|---|---|
| AUC₀₋ₗₐₛₜ | 25.83 | (17.44-38.24) | 69.35 | (46.85-102.68) | 113.92 | (76.95-168.66) | 57.59 |
| AUC_{0-∞} | 47.36 | (32.18-69.71) | 85.15 | (64.54-112.34) | 103.37 | (77.44-137.98) | 28.88 |
| Cₘₐₓ | 154.38 | (122.20-195.05) | 221.59 | (175.39-279.95) | 159.13 | (125.95-201.05) | 32.72 |

Treatment A: 1 × Formula B capsule (THC/CBD: 2.5 mg/2.5 mg) [total dose: 2.5 mg/2.5 mg]
Treatment B: 2 × Formula B capsules (THC/CBD: 2.5 mg/2.5 mg) [total dose: 5 mg/5 mg]
Treatment C: 1 × 1 mL, grapeseed oil containing THC/CBD (10 mg/10 mg) [total dose: 10 mg/10 mg]
Reference: 4 × 100 μL of conventional cannabis extracts containing THC/CBD total dose: 10.8 mg/10 mg]

As shown in Table 6, intra-subject variability of THC was 30.33% for AUC₀₋ₗₐₛₜ, 24.28% for AUC_{0-∞}, and 29.57% for Cₘₐₓ. Intra-subject variability of 11-OH-THC was 25.53% for AUC₀₋ₗₐₛₜ, 23.51% for AUC_{0-∞}, and 26.24% for Cₘₐₓ. Intra-subject variability of CBD was 57.59% for AUC₀₋ₗₐₛₜ, 28.88% for AUC_{0-∞}, and 32.72% for Cₘₐₓ. The non-parametric analysis (Wilcoxon two-sample test, two-sided normal [Z] approximation) of Tₘₐₓ under fasting conditions detected a significant difference in Tₘₐₓ between treatments with Formulation B and with conventional Cannabis extracts.

Since the therapeutic dose of THC is highly variable between patients, it is important that patients can accurately control their dose to get an adequate therapeutic response while also avoiding intolerable side effects. As shown in Table 6 above, individual subject plasma concentration data and pharmacokinetic parameters show a high degree of inter-subject variability following administration of conventional Cannabis extracts.

### Example 4

Randomized, multi-site, double-blind placebo-controlled studies evaluating the effectiveness of Formulation B for the secondary prevention of CINV in patients receiving moderate-to-highly emetogenic intravenous chemotherapy are being conducted in two stages. First is a phase 2 study having a cross-over design to determine feasibility, followed by a phase 3 trial having a parallel group design.

In particular, these studies evaluate whether an anti-emetic regimen incorporating the present oral Formulation B is more effective than a guideline-consistent anti-emetic regimen for the secondary prevention of CINV. Guideline-consistent anti-emetic regimens refer to local, national, or international anti-emetic guidelines (e.g., NCCN, ASCO, and MASCC) that provide recommendations to guide optimal use of anti-emetics.

For the phase 2 study, patients will initially be randomized to one of two treatment groups (placebo or Formulation B) in a ratio of 1:1. Following the first cycle (cycle A) of chemotherapy, patients initially randomized to the placebo group will receive Formulation B, and patients initially randomized to the Formulation B group will receive placebo. Following the second cycle (cycle B) of the phase 2 study is the third chemotherapy cycle (cycle C), during which patients will receive their preferred choice of either placebo or Formulation B. Patients remain blinded until after cycle C.

During each cycle of study treatment (cycles A, B, and C) for the phase 2 and phase 3 studies, participants will receive either oral Formulation B capsules or oral placebo three times per day on the day prior to chemotherapy; prior to and at completion and 4 hours after completion of chemotherapy infusion on day 1 of chemotherapy; then three times per day until the evening of day 5 of chemotherapy, as per Table 7 below. As with many psychoactive medications, oral THC/CBD doses need to be titrated according to tolerance of neurologic and psychiatric adverse effects and nausea control, particularly early in treatment as patients become tolerant to the effects of the medication.

TABLE 7

| | | | | |
|---|---|---|---|---|
| | | | | Maximum dose |
| Day (24 hour | | | | per 24 hour |
| period) | Time | Dose | Dose Titration | period |

Day Prior to Chemotherapy

| | | | | |
|---|---|---|---|---|
| −1 | morning | 1 capsule | N/A | 5 capsules (12.5 mg |
| −1 | afternoon (at | 2 capsules | Miss dose if intoxicated | THC/CBD or |
| | least 4 hours | | | placebo) |
| | after previous | | | |
| | dose) | | | |
| −1 | night (at least 4 | 1-2 capsules | Miss dose if intoxicated; | |
| | hours after | | Reduce dose by 1 capsule if | |
| | previous dose) | | previous dose not tolerated; | |
| | | | Maintain dose if previous dose | |
| | | | tolerated | |

Day 1 of chemotherapy

| | | | | |
|---|---|---|---|---|
| 1 | 60 minutes prior | 1-2 capsules | Miss dose if intoxicated; | 8 capsules (20 mg |
| | to | | Reduce dose by 1 capsule if | THC/CBD or |
| | chemotherapy | | previous dose not tolerated; | placebo) |
| 1 | Immediately | 1-3 capsules | Maintain dose if previous dose | |
| | after completion | | tolerated, with nausea control; | |
| | of day 1 | | Increase dose by 1 capsule if | |
| | chemotherapy | | previous dose tolerated, but | |
| 1 | 4 hours after | 1-3 capsules | inadequate nausea control | |
| | completion of | | | |
| | day 1 | | | |
| | chemotherapy | | | |

After day 1 of chemotherapy

| | | | | |
|---|---|---|---|---|
| 2 | Continue three | 1-4 capsules | Miss dose if intoxicated; | 12 capsules (30 mg |
| 3 | times/day | three times/day | Reduce dose by 1 capsule if | THC/CBD |
| 4 | | | previous dose not tolerated; | or placebo) |
| 5 | Morning | 1-4 | Maintain dose if previous dose | 8 capsules (20 mg |
| | Take final dose on | capsules/dose | tolerated, with nausea control; | THC/CBD or |
| | afternoon of day 5 | | Increase dose by 1 capsule if | placebo) |
| | | | previous dose tolerated, but | |
| | | | inadequate nausea control | |

The primary objective of both studies is to compare, among patients randomized to oral Formulation B or placebo (or within patients in the phase 2 study), the ability to control emesis and nausea. To assess this objective, the primary endpoint is complete response to study medication during the overall phase of 0-120 hours of chemotherapy cycle A (cycles A and B in the phase 2 study), where complete response is defined as no emesis and no use of rescue medication. Control of emesis and nausea will be measured with a patient diary, as is standard for clinical trials of anti-emetics for the prevention of CINV.

Additional endpoints relate to the control of emesis and nausea by assessing the proportion of patients during acute (0-24 hours), delayed (24-120 hours) and overall (0-120 hours) phases of chemotherapy cycles A, B, and C having (i) complete response, (ii) no emesis (vomiting or dry retching); (iii) no significant nausea; and (iv) no use of rescue medications. An additional endpoint includes assessing the number of emetic episodes during 0-120 hours of chemotherapy cycles A, B, and C.

To be included in the studies, patients had to have experienced significant CINV during a previous chemotherapy cycle. Significant CINV is defined as the need for at least one dose of rescue medications for vomiting or distress by nausea, and/or greater than or equal to moderate nausea on a 5-point rating scale (0=nil; 1=minimal; 2=moderate; 3=severe; 4=very severe), despite best-practice eviQ and/or MASCC guideline-consistent anti-emetic regimens, at any time from Treatment Day 1 to the end of the planned duration of the previous cycle.

The estimated sample size for the pilot phase 2 trial is 80 patients, using a primary endpoint of complete response to study drug and placebo during cycle A and B of study treatment. Using a cross-over design, a sample size of 80 patients, and randomizing patients to either study drug followed by placebo or placebo followed by study drug, will have 80% power at a 2-sided significance level of 10% to detect a 20% difference in discordant responses (response on one intervention but not the other). Accrual is expected to take 12 months. The primary analysis will be a comparison of the proportion of patients with complete response between the two treatment arms over two overall phases of chemotherapy cycles (cycle A and cycle B), using a McNemar's test.

The estimated sample size for the definitive phase 3 trial is 250 patients (125 per arm), using a primary endpoint of complete response during cycle A of chemotherapy. Patients in the phase 3 trial will remain in the same treatment group (either placebo or Formulation B) throughout all three chemotherapy cycles. A sample size of 250 patients provides 80% power at 2-sided 5% level of significance to detect improvement in complete response from 22% to 42.5%. Accrual is expected to take 2.5 years. The primary analysis will be a comparison of the proportion of patients with complete response in the two treatment arms during the overall phase of cycle A, using a Chi-square test.

Analysis for each trial will occur after a minimum follow-up of 30 days for all patients. The sample size for each trial allows for a drop-out/ineligibility rate of 20%. The 20% difference is based on the assumption that 42% of patients on study drug will respond compared to 22% on placebo and that the responders in the placebo group will respond/not respond equally on the study drug (11% respond on each). The current evidence suggests that this level of improvement is both worthwhile and feasible. Due to this a pilot phase 2 study in a limited number of patients is being conducted to determine if the activity (improvement in complete response), tolerability of the product (rate of serious adverse events), and accrual rate to the trial, is sufficient to justify completing the definitive phase 3 trial.

Chemotherapy agents classified as low and/or minimal emetic risk can be used concurrently throughout the treatment period for both trials. Acceptable combinations include, but are not limited to, FOLFOX, carboplatin Day 1 and gemcitabine Days 1 and 8. Multi-day use of chemotherapy agents of high or moderate emetic risk is permitted up until but not beyond Day 5, when the continuation of the chemotherapy is part of the overall Day 1 regimen. Acceptable multi-day regimens include, but are not limited to: weekly cisplatin, weekly carboplatin, cisplatin Days 1 and 8 with gemcitabine Days 1 and 8. Patients are able to commence additional permitted regular and/or rescue anti-emetic therapy at the time of trial entry at the physician's discretion.

Patients will also be allowed to take pre-specified rescue medications for nausea or vomiting throughout both trials. Patients will be provided with a prescription for rescue therapies according to investigator selection. Permitted rescue therapies include: Lorazepam (e.g., 1 mg PO bd prn); Metoclopramide (e.g., 10 mg taken orally, three times per day as needed; Haloperidol (e.g., 0.5-1 mg taken orally, three times per day as needed); Prochlorperazine (e.g., 5-10 mg taken orally, three times per day as needed; or 25 mg suppositories taken rectally, every eight hours as needed); or Olanzapine (e.g., 5 mg taken orally two times per day, or 10 mg taken orally every morning for three days).

Patients also receive anti-emetic prophylaxis with a 5HT₃ antagonist, steroid, and (where indicated) NK1 antagonist, according to a prespecified choice of regimen. A summary of treatment administration is provided below in Table 8.

TABLE 8

| Day | −1 | 1 | 2 | 3 | 4 | 5 |
|---|---|---|---|---|---|---|
| Chemotherapy | | X | R | R | R | R |
| Oral Formulation B or placebo | X | X | X | X | X | X |
| 5HT₃ antagonist | | X | R | R | | |
| Dexamethasone | | X | X | X | R | R |
| NK1 antagonist | | R | | | | |
| Rescue medications | | 0 | 0 | 0 | 0 | 0 |

X = drug administered
R = regimen-dependent
0 = as required
Treatment Duration: Up to 9 weeks (3 consecutive cycles of chemotherapy)

While some preferred embodiments of the invention have been described, it should be understood that various changes, adaptations and modifications may be made therein without departing from the spirit of the invention and the scope of the appended claims. It is intended that all such modifications and alterations be included insofar as they come within the scope of the invention as claimed or the equivalents thereof.

What is claimed is:

1. A method of treating chemotherapy-induced nausea and vomiting by administering to a patient in need thereof a formulation comprising pharmaceutically effective amounts of cannabidiol and delta-9-tetrahydrocannabinol, wherein a weight ratio of the cannabidiol to the delta-9-tetrahydrocannabinol in the formulation is from 2:0:0.5 to 0.5 to 2.0, wherein the formulation comprises a solid oral dosage form, and wherein the administering occurs before and/or after the patient receives chemotherapy treatment.

2. The method of claim 1 wherein the weight ratio of the cannabidiol to the delta-9-tetrahydrocannabinol in the formulation is about 1:1.

3. The method of claim 1 wherein the pharmaceutically effective amount of the cannabidiol is between about 2.5 mg/day and about 30 mg/day, and the pharmaceutically effective amount of the delta-9-tetrahydrocannabinol is between about 2.5 mg/day and about 30 mg/day.

4. The method of claim 1 wherein the chemotherapy-induced nausea and vomiting is acute chemotherapy-induced nausea and vomiting, delayed chemotherapy-induced nausea and vomiting, or both acute and delayed chemotherapy-induced nausea and vomiting.

5. The method of claim 1 wherein the patient does not experience any nausea from the chemotherapy treatment.

6. The method of claim 1 wherein the patient does not experience any vomiting from the chemotherapy treatment.

7. The method of claim 1 wherein the formulation is an oral liposomal formulation.

8. The method of claim 1 wherein the formulation is administered to the patient one day before, on a day of, or one or more days after, the patient receives the chemotherapy treatment.

9. The method of claim 8 wherein on the day before the patient receives the chemotherapy treatment, the patient is administered between about 2.5 mg and about 12.5 mg of the cannabidiol and between about 2.5 mg and about 12.5 mg of the delta-9-tetrahydrocannabinol.

10. The method of claim 8 wherein on the day the patient receives the chemotherapy treatment, the patient is administered between about 2.5 mg and about 20 mg of the cannabidiol and between about 2.5 mg and about 20 mg of the delta-9-tetrahydrocannabinol.

11. The method of claim 8 wherein on the day the patient receives the chemotherapy treatment, the formulation is administered to the patient immediately after the chemotherapy treatment.

12. The method of claim 8 wherein on the day the patient receives the chemotherapy treatment, the formulation is administered to the patient both before and after the chemotherapy treatment.

13. A formulation comprising cannabidiol and delta-9-tetrahydrocannabinol, wherein the formulation is a solid oral dosage form configured to be swallowed by a patient, and a weight ratio of the cannabidiol to the delta-9-tetrahydrocannabinol in the formulation is from 2:0:0.5 to 0.5 to 2.0.

14. The formulation of claim 13 wherein the formulation is an oral liposomal formulation.

15. The formulation of claim 13 wherein the formulation comprises about 2.5 mg of the cannabidiol and about 2.5 mg of the delta-9-tetrahydrocannabinol.

16. The formulation of claim 13 wherein the weight ratio of the cannabidiol to the delta-9-tetrahydrocannabinol in the formulation is about 1:1.

17. The formulation of claim 13 further comprising one or more of TPGS and PHOSPHOLIPON 85G.

18. The formulation of claim 13 further comprising orange oil.

19. The formulation of claim 13 further comprising ethanol.

20. The formulation of claim 13 further comprising glycerin.
